(19) United States
(12) Patent Application Publication
(10) Pub. No.: US 2008/0228180 A1
(43) Pub. Date: Sep. 18, 2008

(54) Ablation system and heat preventing electrodes therefor

(75) Inventor: Gordon Epstein, Fremont, CA (US)

Correspondence Address: BROWN RUDNICK LLP, ONE FINANCIAL CENTER, BOSTON, MA, 02111, US

(73) Assignee: Halt Medical, Inc, Pleasanton, CA (US)

(21) Appl. No.: 11/717,920

(22) Filed: Mar. 13, 2007

(51) Int. Cl. A61B 18/14

(52) U.S. Cl. 606/34

(57) ABSTRACT

An ablation system comprising a source of electrical ablation energy having first, second and third power outputs it is disclosed. A first conductor is coupled to the first power output on the source of electrical energy. A second conductor is coupled to the second power output on the source of electrical energy, the source of electrical energy creates a first output ablation voltage between the first and second power outputs. The first output ablation voltage varies between a first higher average value during a first period of time and a first lower average value for a second period of time. The first lower average value is greater than or equal to zero. A third conductor is coupled to a third power output on the source of electrical energy. The source of electrical energy creates a second output ablation voltage between the first and third power outputs. The second output ablation voltage varies between a second higher average value during a third period of time and a lower average value for a fourth period of time. The lower average value is greater than or equal to zero. An ablation probe is coupled to the first conductor.

### TECHNICAL FIELD

The present invention relates to the field of electrodes which are applied to the skin for the purpose of providing a return current path for an ablation system.

### BACKGROUND

Ablation is a recognized method for the treatment of certain lesions. These include cancerous and “benign” growths, such as lesions in the liver, as well as other growths, such as uterine fibroids. The treatment of uterine fibroids is discussed in U.S. Pat. No. 6,840,935 of Dr. Bruce Lee, dated Jan. 11, 2005, and directed toward a gynecological ablation procedure and system using an ablation needle. The system of the present invention is well-suited to gynecological ablation procedures.

A uterine fibroid ablation procedure requires up to about two amperes of RF current. That current represents the delivery of 150 watts into a load of about 40 ohms. Typically, a pair of electrode pads are used in order to attain the current flow needed without undesirable side effects, such as electrode heating. Thus, with two return electrode pads about one ampere will flow in each electrode pad, if the current flow is perfectly balanced. More likely, there will be some imbalance, so current in excess of one ampere through a particular electrode pad is likely.

Heating in the vicinity of the pads is typical, and overheating is usually a concern. Skin temperature will increase depending upon power dissipated under the electrode pad. Power dissipated under the electrode pad is directly proportional to power applied. Heat increases with increased power and increased procedure time. Since P=I²R, temperature increases approximately as the square of current. Where one employs high current and long procedure times, overheating is of particular concern.

The liver lesion ablation procedure most commonly employed using an ablation apparatus manufactured by Rita Medical can involve currents and times comparable to a uterine fibroid ablation procedure, and thus share similar overheating problems. As alluded to above, ablation relies upon the application of electrical energy between, for example, a trocar carrying a plurality of ablation stylets and a return electrode or electrodes. Prior art ablation procedures call for “icing” the return electrodes. Failure to do so may result in patient burns.

Prior art electrodes incorporate a single thermocouple for monitoring skin temperature to address the problem of overheating and resultant burns.

### SUMMARY OF THE INVENTION

In accordance with the invention, an ablation system comprises a source of electrical ablation energy having first, second and third power outputs. A first conductor is coupled to the first power output on the source of electrical energy. A second conductor is coupled to the second power output on the source of electrical energy, the source of electrical energy creates a first output ablation voltage between the first and second power outputs. The first output ablation voltage varies between a first higher average value during a first period of time and a first lower average value for a second period of time. The first lower average value is greater than or equal to zero. A third conductor is coupled to a third power output on the source of electrical energy. The source of electrical energy creates a second output ablation voltage between the first and third power outputs. The second output ablation voltage varies between a second higher average value during a third period of time and a lower average value for a fourth period of time. The lower average value is greater than or equal to zero. An ablation probe is coupled to the first conductor.

An electrode provides a return path for an ablation device. The electrode comprises a first conductive ablation member which defines a first contact surface. The first contact surface defines a first active peripheral edge on a first active side of the first conductive member. A first coupling member is coupled to the second conductor and electrically connected to a first power coupling edge of the first conductive member. The second edge is an edge of the first conductive member other than the first active peripheral edge. The first coupling member is made of an electrically conductive material. A second conductive ablation member and defines a second contact surface, the second contact surface defines a second active peripheral edge on a second active side of the second conductive member. A second coupling member coupled to the third conductor is electrically connected to a second power coupling edge of the second conductive member. The second power coupling edge is an edge of the second conductive member other than the second active peripheral edge. The second coupling member is made of an electrically conductive material. The first active peripheral edge is positioned between the first power coupling edge and the second power coupling edge.

In accordance with the inventive system, the first period of time may overlap a substantial portion of the fourth period of time, and the second period of time may overlap a substantial portion of the third period of time.

In accordance with a preferred embodiment of the invention, the power coupling edges may be opposite the active peripheral edges.

In a preferred embodiment, the peripheral edges are substantially straight and have first and second ends, and a curved edge is contiguous to each of the first and second ends.

In accordance with the inventive method of ablating a biological body in a mammal, ablation energy is applied between an ablation stylet and, intermittently, first and second skin electrodes.

### BRIEF DESCRIPTION OF THE DRAWINGS

The invention will be understood from the description presented below, taken together with the drawings, in which:

FIG. 1 illustrates electrode placement on the thighs of a subject;

FIG. 2 is a diagram illustrating the heating and cooling of the body adjacent a return electrode pad;

FIG. 3 is a schematic representation of a known return path electrode pad useful in connection with the present invention;

FIG. 4 is a schematic representation of an alternative return path electrode pad useful in connection with the present invention;

FIG. 5 is a schematic representation of another alternative return path electrode pad useful in connection with the present invention; and

FIG. 6 is a schematic representation of still another alternative return path electrode pad useful in connection with the present invention.

### DETAILED DESCRIPTION OF THE PREFERRED EMBODIMENT

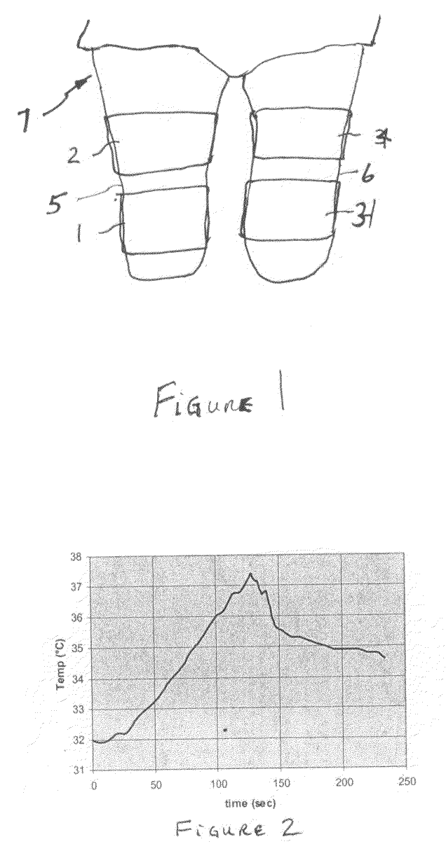

In accordance with the present invention, electrodes, for example any one of electrodes 1-4 illustrated in FIG. 1, having a size of, for example, 12.5 cm in width and 25 cm in length, are applied to the thighs 5 or 6 of a human subject 7. More particularly, in accordance with the present invention, applied power to the electrodes is multiplexed with one electrode receiving power while the others not receiving power and alternative the application of power to the return electrodes. In accordance with the preferred embodiment power is continuously applied to the ablation electrode, with power being applied to each of the electrodes individually for only a portion of that period of time during which power is applied to the ablation electrode.

Upon the application of a current to the electrodes, heating, over a period of approximately 130 seconds was noted as illustrated in FIG. 2. Upon the removal of the current, cooling occurred as illustrated in FIG. 2, with initial cooling being more rapid than heating or long-term cooling.

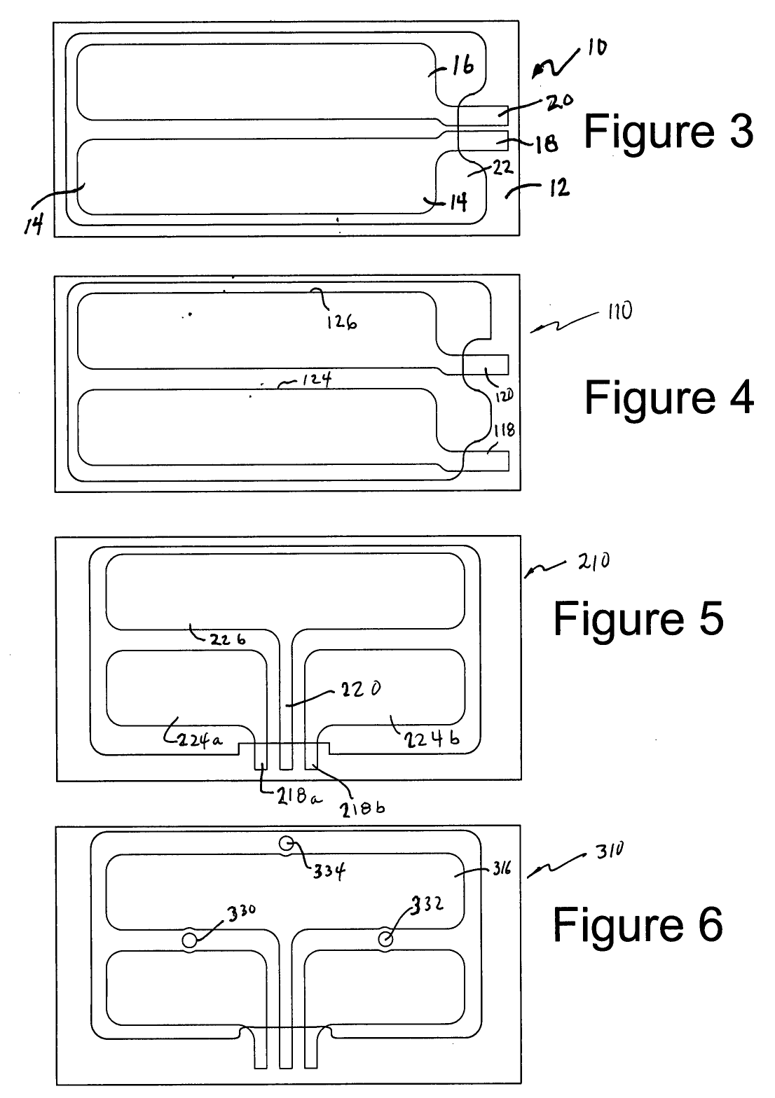

Preliminary tests involved application of Aaron Medical (Bovie) ESRE-1 return electrodes, similar to those illustrated in FIG. 3, to the anterior of the two thighs for test purposes only. That is to say, current was applied between electrodes for test purposes, rather than between an electrode or electrodes and an ablation needle. Tests were conducted on three different subjects (1 female, 2 male). RF current at approximately 460 kHz, produced by a Rita Medical ablation system was used.

Referring to FIG. 3, a return electrode suitable for use in accordance with the method of the present invention is illustrated. Electrode 10 comprises a base 12 a pair of electrodes 14 and 16. A lead 18 is associated with and integral electrode 14, optionally being stamped from a single sheet of conductive material, such as copper. A lead 20 is associated with and integral with electrode 16, optionally being stamped from a single sheet of conductive material, such as copper.

Electrodes 14 and 16 are held in position by an adhesive layer mounted on a support member 22. Support member 22 may be made of fabric, plastic or any other suitable material. As illustrated in FIG. 3, base 12 is coated with a release material which adheres to the adhesive layer supported on support member 22. Before use base 12 is removed, exposing the adhesive layer. Electrodes 14 and 16 are adhered to the same layer of adhesive which adheres to the skin of the thigh, for example, of a subject, and brings the electrodes 14 and 16 into contact with, for example, the thigh of the subject.

During a test, using an electrode similar to that illustrated in FIG. 3, resistance was first measured between the two halves of the split electrode, namely electrodes 14 and 16. The resistance from one electrode half 14 to the other electrode half 16 was approximately 52 ohms for the female subject and 20 to 24 ohms for the male subjects. When measuring the resistance from an electrode on one leg to an electrode on the other leg, the total resistance was measured at approximately 79 ohms for the female subject and 67 to 68 ohms for the male subjects.

It appears that it may be difficult to determine how well the return electrode contacts are made by measuring resistance from one pad to the other (one leg to the other). However, in the relatively small sample tested, the difference from leg to leg was only about 11 to 12 ohms (about 15% of the total leg to leg resistance). Yet the resistances between the pad halves differed by more than a factor of two. Accordingly, it is believed that measuring the resistance between the halves of a split electrode is a better indicator of contact integrity.

The resistances involved at the pad sites and between the pads and the procedure location (the point at which the monopolar ablation electrode is applied) thus appear to be a significant portion of the total resistance. Thus, only a fraction of the total power applied is available to do the intended work at the procedure location.

In a series of tests conducted with the Rita Medical RF source, a Flir Systems A40M infrared camera was used to monitor temperature.

Resistance from pad-half to pad-half was measured at 19 to 24 Ohms (about the same as measured previously on this subject) Resistance measured from left leg to right leg was measured at approximately 60 Ohms (slightly lower than measured previously)

Referring to FIG. 1, two ESRE-1 pads were placed on each leg, forming an array of four electrodes (electrode 1 on one thigh nearest the foot, electrode 2 nearest the torso on the same thigh, and electrode 3 nearest the foot and electrode 4 nearest the torso on the other thigh). The resistance between immediately adjacent electrodes (electrode 1 and electrode 2 in one case, and electrode 3 and electrode 4 in another) was 23 to 24 Ohms. The resistance between alternate electrodes (electrode 1 and electrode 4 or electrode 2 and electrode 3) was 36 to 40 Ohms. With the 4-electrode array just described, a current of 2 Amps between alternate electrodes (electrode 2 and electrode 4) could not be tolerated for more than a few seconds. Discomfort was felt in the vicinity of the cable connections.

At 1.4 Amps, the subject could tolerate the application of current for a bit longer, but not for more than about 30 seconds. When 1.4 Amps was applied for 15 seconds, and then off for 15 seconds, this procedure could be tolerated for 4 minutes or more. This apparently gives the tissue under the electrode being used time to cool down. While the subject felt discomfort in the area under the electrode pad connections (such as leads 18 and 20), the thermal camera did not indicate that there was more heating adjacent the electrode that connections. However, it may be that some subcutaneous effects were being felt. It is noted that this was with ESRE-1 electrode pads oriented laterally, i.e., as illustrated in FIG. 1.

As illustrated in FIG. 2, when an RF current of approximately 0.7 Amp was applied between immediately adjacent electrodes (e.g., electrode 1 and electrode 2, the rate of rise of temperature over time was observed to be less than the rate of fall, at least over some time interval, indicating that time multiplexing the electrodes has a beneficial effect heating.

In the test illustrated in FIG. 2, an RF current (0.7 Amps) was turned on for about 10-20 seconds, and was allowed to run for 2 minutes. The current was then turned off and the skin temperature allowed to return toward equilibrium. The initial skin temperature was about 32° C. The temperature rose linearly at about 0.05° C./second. After the RF current was turned off, the temperature fell at about 0.087° C./second for about 15 seconds. The temperature did not return to the original (32° C.) value until long after the current flow stopped.

It was observed that when current is applied from one pad to the other the hottest parts are along the line between the pads. There is considerably less heating in portions of the electrode progressively further from the edge of the pad that is in closest proximity to the other part of the circuit.

Generally, it was observed that the portion of the edge (the “leading edge”) of an electrode closest to the other electrode (which simulates the ablation needle) conducts substantially most of the current, thus resulting in that edge developing considerable heat in the adjacent portion of the skin.

The problem with the configuration illustrated in FIG. 3 is that the electrical connections are at one side. If the upper part is conducting, the “leading edge” is away from the connection point. However, if current is flowing to the lower part, the leading edge is along the line that includes the connection part or lead. This appears to contribute to the discomfort felt by the subject.

An alternative electrode pad 110 with a different configuration is illustrated in FIG. 4. Here, leading edges 124 and 126 are opposite leads 118 and 120, promoting patient comfort.

In yet another alternative electrode 210, illustrated in FIG. 5, lower left and right lower electrode sections 224a and 224b, as well as electrode 226 are connected together (by a conductor in a conventional clamp connector) to effectively form a single long electrode. This is done because the client connector grasps all three leads 218a, 218b and 220.

Referring to FIG. 6, yet another alternative electrode 310 is illustrated. In this embodiment, three spots are provided for thermocouples 330, 332, and 334. Thermocouple 334 is provided at the center of the upper part, and thermocouples 330 and 332 are provided along the leading edge of the lower part of the electrode 316. This configuration has the advantage of monitoring the temperature along each electrode edge, and also monitoring at three positions laterally. If the electrode pad his being “iced” but the ice pack it is not being applied uniformly, there is thus a better chance of detecting the error and alerting the physician.

While an illustrated embodiment of the invention has been described, it is, of course, understood that various modifications will be obvious to those of ordinary skill in the art. Such modifications or within the spirit and scope of the invention which is limited and defined only by the appended claims.

1. An ablation system for ablating a biological mass, comprising:
(a) a source of electrical ablation energy having first, second and third power outputs;
(b) a first conductor coupled to said first power output on said source of electrical energy;
(c) a second conductor coupled to said second power output on said source of electrical energy, said source of electrical energy creating a first output ablation voltage between said first and second power outputs, said first output ablation voltage varying between a first higher average value during a first period of time and a first lower average value for a second period of time, said first lower average value being greater than or equal to zero;
(d) a third conductor coupled to a third power output on said source of electrical energy, said source of electrical energy creating a second output ablation voltage between said first and third power outputs, said second output ablation voltage varying between a second higher average value during a third period of time and a lower average value for a fourth period of time, said lower average value being greater than or equal to zero;
(e) an ablation probe, said ablation probe being coupled to said first conductor;
(f) a first return electrode having a first elongated edge coupled to a first drive electrode portion;
said first drive electrode portion being coupled to said second conductor and said first elongated edge being positioned between said first drive electrode portion and said biological mass; and
(g) a second return electrode having a second elongated edge coupled to a second drive electrode portion: said second drive electrode portion being coupled to said third conductor and said second elongated edge being positioned between said second drive electrode portion and said biological mass.

2. An ablation system as in claim 1, wherein said first period of time overlaps a substantial portion of said fourth period of time, and said second period of time overlaps a substantial portion of said third period of time.

3. An ablation system as in claim 1, wherein said first electrode for providing a return path for said ablation probe, comprises a first conductive member defining a first contact surface, said first contact surface defining a first active peripheral edge on a first active side of said first conductive member, a first coupling member coupled to said second conductor and electrically connected to a first power coupling edge of said first conductive member, said second edge being an edge of said first conductive member other than said first active peripheral edge, said first coupling member being made of an electrically conductive material, and, said second electrode for providing a return path comprises a second conductive member defining a second contact surface, said second contact surface defining a second active peripheral edge on a second active side of said second conductive member, a second coupling member coupled to said third conductor and electrically connected to a second power coupling edge of said second conductive member, said second power coupling edge being an edge of said second conductive member other than said second active peripheral edge, said second coupling member being made of an electrically conductive material, said first active peripheral edge being positioned between said first power coupling edge and said second power coupling edge.

4. An ablation system as in claim 3, wherein said first period of time overlaps a substantial portion of said fourth period of time, and said second period of time overlaps a substantial portion of said third period of time.

5. An ablation system as in claim 4, wherein said power coupling edges are opposite said active peripheral edges.

6. An ablation system as in claim 4, wherein said peripheral edges are substantially straight and have first and second ends, and wherein a curved edge is contiguous to each of said first and second ends.

7. An ablation system as in claim 4, wherein a curved edge is contiguous to each of said first and second ends.

8. An ablation system as in claim 4, wherein said peripheral edges are substantially straight and have first and second ends.

9. An ablation system as in claim 4, wherein said first and second contact surfaces are coated wit an adhesive.

10. An ablation system as in claim 4, wherein said first conductive member defining a first contact surface defines a two portion contact surface, each defining a portion of said a first active peripheral edge, said two portion contact surface defining a space between said two portions of said contact surface, said second power coupling edge being connected to said third conductor by a conductive strip positioned between said two portions of said contact surface.

11. An ablation system, comprising:
(a) a source of electrical ablation energy having first, second and third power outputs;
(b) a first conductor coupled to said first power output on said source of electrical energy;
(c) a second conductor coupled to said second power output on said source of electrical energy, said source of electrical energy creating a first output ablation voltage between said first and second power outputs, said first output ablation voltage varying between a first higher average value during a first period of time and a first lower average value for a second period of time, said first lower average value being greater than or equal to zero;
(d) a third conductor coupled to a third power output on said source of electrical energy, said source of electrical energy creating a second output ablation voltage between said first and third power outputs, said second output ablation voltage varying between a second higher average value during a third period of time and a lower avenge value for a fourth period of time, said lower average value being greater than or equal to zero;
(e) an ablation probe, said ablation probe being coupled to said first conductor; and
(f) a skin contacting electrode, comprising:
(i) a first electrode portion coupled to said second conductor;
(ii) a second electrode portion coupled to said third conductor.

12. An ablation system, comprising:
(a) a source of electrical ablation energy having first and second power outputs;
(b) a first conductor coupled to said first power output on said source of electrical ablation energy;
(c) a second conductor coupled to said second power output on said source of electrical ablation energy, said source of electrical ablation energy creating an output ablation voltage between said first and second power outputs, said output ablation voltage varying between a higher average value during a first period of time and a lower average value during a second period of time, said lower average value being greater than or equal to zero;
(d) an ablation probe, said ablation probe being coupled to said first conductor; and
(e) a skin contacting electrode, coupled to said second conductor.

13. A method of ablating a biological body in a mammal, comprising applying intermittent ablation energy between an ablation stylet and a skin electrode.

14. A method of ablating a biological body in a mammal, comprising applying ablation energy between an ablation stylet and, intermittently, first and second skin electrodes.

15. A method of ablating a biological body in a mammal as in claim 14, wherein ablation energy is applied between an ablation stylet and, alternately, first and second skin electrodes.
